(19) United States
(12) Patent Application Publication
(10) Pub. No.: US 2005/0063917 A1
(43) Pub. Date: Mar. 24, 2005

(54) Solid oral compositions

(76) Inventors: Roberto Colle, Lainate (IT); Guglielmo Salmoiraghi, Lainate (IT); Andrea Barrica, Lainate (IT)

Correspondence Address: YOUNG & THOMPSON, 745 SOUTH 23RD STREET, 2ND FLOOR, ARLINGTON, VA, 22202, US

(21) Appl. No.: 10/494,567

(22) PCT Filed: Nov. 5, 2002
(86) PCT No.: PCT/EP02/12330

(30) Foreign Application Priority Data
Nov. 6, 2001 (IT) MI2001A002322

(51) Int. Cl. A61K 9/68; A61K 35/78

(52) U.S. Cl. 424/48; 424/748; 424/737; 424/757; 424/769

(57) ABSTRACT

Oral formulations in the form of chewing gum comprising: a. polyphosphates; b. hydrated silica; c. a source of fluoride ions; d. extracts or active ingredients of vegetable origin; e. optionally antibacterial/disinfectant agents. The compositions according to the invention are useful as adjuvants in dental hygiene, in particular to reduce tartar deposits.

The present invention relates to oral anti-tartar and anti-plaque compositions, useful as adjuvants in odontostomatological hygiene.

### BACKGROUND TO THE INVENTION

The problem of dental plaque and tartar formation has long been studied, and agents which can be used to combat and delay such formation are being actively researched.

The mechanisms that cause tartar deposits are well known; these deposits are constituted by calcium phosphate crystals which precipitate in the extracellular matrix of bacterial plaque. The pathogenetic role of tartar in periodontal diseases such as pyorrhoea, periodontitis, gingivitis and correlated disorders is equally well known.

Various substances have proved effective in reducing or preventing tartar formation and deposits on the teeth, including soluble pyrophosphates and polyphosphates, zinc salts, fluorides, diphosphonates, antibacterial agents such as triclosan, and abrasive agents such as silica or alumina. These substances, combined with one another in various ways, are included in the composition of most anti-tartar toothpastes now commercially available. The clinical efficacy of these toothpastes has been examined in numerous studies, reviewed in J. Clin. Dent. Vol IV(3), 71-81, 1993.

The most common toothpastes contain soluble polyphosphates associated with fluorides and silica, and possibly with polymers that possess bioadhesive properties, as described, for example, in U.S. Pat. No. 4327977, U.S. Pat. No. 4,889,713, U.S. Pat. No. 5,017,362, U.S. Pat. No. 5,139,769, U.S. Pat. No. 4,921,693 and EP 492997.

Similar compositions, with the addition of antibacterial agents such as triclosan, are described, for example, in GB 2200551. In addition to toothpastes, chewing gums and candies with a similar composition have been developed.

The efficacy of these toothpastes, which has been the object of numerous studies (J. Clin. Dent. Vol. X(3), 99-102, 1999; Oral Surg. Oral Med. Oral Pathol., Vol 70(4), 529-536, 1990; J. Clin. Dent. Vol. IX(4), 101-104, 1998), is due to inhibition of calcium phosphate precipitation by the polyphosphates that complex the calcium ions in the saliva, to the abrasive action of silica, to the reinforcing effect of fluorides on the tooth enamel and to the action of bioadhesive polymers, where used, which protects the mucosae and causes slow release of the other ingredients.

Candies and chewing gum designed as adjuvants in dental hygiene and oral hygiene in general, which have properties that can be described as anti-tartar, anti-decay, whitening and/or refreshing, are becoming increasingly popular. The main advantage of these forms of administration is that they can be used freely and conveniently during the day in any place and on any occasion, in addition to that fact that the release of the active elements (functional ingredients) is slower and more regular than in the case of an ordinary toothpaste.

### DESCRIPTION OF THE INVENTION

The present invention relates to oral formulations in a solid form, preferably in the form of chewing gum, whose efficacy is superior to that of similar known formulations.

The compositions of the invention contain effective amounts of:
a. polyphosphates, preferably a mixture of alkali metal pyrophosphates and triphosphates;
b. an abrasive agent (preferably hydrated silica);
c. a source of fluoride ions;
d. extracts or active ingredients of vegetable origin;
e. optionally antibacterial or disinfectant agents.

In addition to the active ingredients referred to above, the compositions of the invention will contain excipients suitable to define the final form of administration.

Thus, for example, a chewing gum formulation will require a suitable base consisting of gum base, sweeteners, polyalcohols such as xylitol, sorbitol and mannitol, flavourings, dyes, softeners, plasticisers, stabilisers, thickeners, etc.

The polyphosphates used in the compositions may be alkali metal pyrophosphates (diphosphates), hexametaphosphates or tripolyphosphates or mixtures thereof. A mixture of disodium diacid diphosphate and pentasodium or pentapotassium triphosphate is particularly preferred. It has been proved that a toothpaste containing this mixture provides more marked reduction of tartar than a toothpaste containing pyrophosphates not associated with triphosphates (J. Clin. Dent. Vol. IX(4), 101-104, 1998).

Broadly, the polyphosphates amount to between 0.5 and 5% by weight on the total composition of the invention.

The function of the abrasive agent is to increase the plaque-removing action already possessed by ordinary chewing gum. It may be formed by hydrated silica (in a suitable form), calcium carbonate (in a suitable form) or talc, either individually or in a mixture thereof. These abrasives may also be present totally or partly, individually or combined with one another, in encapsulated form, in particular encapsulated in calcium alginate. Chewing gum containing microgranules of hydrated silica encapsulated in calcium alginate has proved more effective in removing plaque than a chewing gum with the same formulation but without microgranules (Doc. Os 06.2001 779-781). According to the present invention, the encapsulated microgranules may contain dyes, flavourings, functional ingredients and herb extracts. This abrasive agent is usually present in percentages of between 0.5 and 7% by weight.

Suitable sources of fluoride ions include sodium fluoride, potassium fluoride, ammonium fluoride, sodium monofluoro-phosphate and other known non-toxic salts containing fluorine, in concentrations which provide fluoride percentages of between 0.005 and 0.2% by weight.

The vegetable extracts which may be present in the compositions of the invention will preferably be selected from extracts of Centella asiatica, Malva sylvestris, Melaleuca alternifolia, Commiphora abyssinica (myrrh), Krameria triandra (rhatany), Acacia catechu, Medicago sativa (alfalfa), resins of the genus Styrax, such as Styrax benzoin (benzoin), Matricaria recutita (camomile), Echinacea purpurea (echinacea) and Croton lechleri (dragon's blood). Extracts of these plants, whose activity has been known for some time, are commercially available.

The combination of these extracts gives the formulations anti-inflammatory/decongestant, emollient, wound-healing, antiseptic and astringent properties. These properties are desirable in at least two respects in the ambit of the present invention:
firstly, to assist and reinforce the reduction in diseases of the oral mucosa caused by the reduction in tartar, and
secondly, to control and prevent contact stomatitis similar to that manifested with the use of toothpastes, known as “toothpaste stomatitis”, in particularly predisposed persons.

In the formulations according to the invention, these extracts may be encapsulated in alginate together with the abrasive agent.

Said extracts may be added to the formulations in percentages of between 0.01 and 2% by weight.

The formulations of the invention can be prepared by conventional techniques, by adding and mixing the various ingredients to the gum base in the case of chewing gum, which may then undergo coating operations in accordance with equally conventional techniques.

The formulations of the invention may include disinfectant or antibacterial agents such as triclosan, zinc salts or zinc oxide, either alone or combined with one another, in concentrations of between 0.1 and 5% by weight. These agents are designed to combat the formation of bacterial plaque, which leads to tartar deposits.

The formulations of the invention may also include decorative crystals, preferably consisting of gum arabic and dyes deposited on the surface of the product with a purely aesthetic function.

Daily use of the chewing gum according to the invention reduces tartar deposits and has other beneficial effects on the condition of the oral and gingival mucosa.

The example below illustrates the invention in greater detail.

### EXAMPLE

Coated Chewing Gum Weighing 1.4 g with Vegetable Extracts.

Percentage Composition (By Weight)

| Ingredient | % |
|---|---|
| Gum base | 25.5 |
| Xylitol | 23.5 |
| Sorbitol | 23.2 |
| Mannitol | 16 |
| Flavouring | 1.8 |
| Silicon dioxide | 3 |
| Gum arabic | 1 |
| Glycerin | 1 |
| Disodium diacid diphosphate | 1 |
| Pentasodium triphosphate | 1 |
| Mallow, myrrh, centella, melaleuca, rhatany and cutch extracts | 0.05 |
| Maltitol syrup | 0.93 |
| Titanium dioxide (E171) | 0.7 |
| Quick Coat | 0.6 |
| Aspartame | 0.6 |
| Acesulfame | 0.05 |
| Carnauba wax | 0.05 |
| Potassium fluoride | 0.02 |
| Total | 100 |

1. Oral formulations in the form of chewing gums, comprising:
a. polyphosphates;
b. an abrasive agent;
c. a source of fluoride ions;
d. extracts of vegetable origin;
e. optionally antibacterial or disinfectant agents wherein the extracts are selected from extracts of centella asiatica, malva sylvestris, melaleuca alternifolia, commiphora abyssinica (myrrh), krameria triandra (rhatany), acacia catechu, medicago sativa (alfalfa), resins of the genus styrax, such as styrax benzoin (benzoin), matricaria recutita (camomile), echinacea purpurea (echinacea) and croton lechleri (dragon's blood).

2. Formulations as claimed in claim 1, wherein the polyphosphates are selected from tripolyphosphates, pyrophosphates or mixtures thereof.

3. Formulations as claimed in claim 2, comprising a mixture of alkali metal pyrophosphates and tripolyphosphates.

4. Formulations as claimed in claim 1, further containing excipients selected from gum base, sweeteners, polyalcohols, flavourings, dyes, softeners, plasticisers, stabilizers and thickeners

5. Formulations as claimed in claim 1, wherein the abrasive agents are hydrated silica, calcium carbonate and talc, either individually or in a mixture thereof.

6. Formulations as claimed in claim 1 containing extracts with anti-inflammatory, wound-healing, antihemorrhagic, soothing, emollient, decongestant and antiseptic properties.

7. Formulations as claimed in claim 1, containing 0.5 to 5% by weight of polyphosphates, 0.5 to 7% by weight of an abrasive agent (possibly wholly or partly encapsulated), a source of fluorine ions able to guarantee a fluoride intake of 0.005 to 0.2%, and 0.01 to 2% by weight of plant extracts.

8. Formulations as claimed in claim 1, comprising disinfectant and/or antibacterial agents selected from triclosan, zinc oxide and zinc salts, either alone or in combination with one another, in concentrations of between 0.1% and 5%.

9-10. (cancelled).
